(19) United States
(12) Patent Application Publication
(10) Pub. No.: US 2017/0291013 A1
(43) Pub. Date: Oct. 12, 2017

(54) GUIDEWIRE

(71) Applicant: BOSTON SCIENTIFIC SCIMED, INC., Maple Grove, MN (US)

(72) Inventors: PETER J. PEREIRA, Mendon, MA (US); STEVEN E. WALAK, Natick, MA (US)

(21) Appl. No.: 15/479,970

(22) Filed: Apr. 5, 2017

Related U.S. Application Data
(60) Provisional application No. 62/319,080, filed on Apr. 6, 2016.

(51) Int. Cl. A61M 25/09

(52) U.S. Cl. CPC A61M 25/09; A61M 2025/09083; A61M 2025/09175; A61M 2025/09133; A61M 2025/09166; A61M 2025/09108

(57) ABSTRACT

A guidewire includes an elongate core member having an intermediate segment, a distal segment and a proximal segment, a distal shoulder between the intermediate segment and the distal segment and a proximal shoulder between the intermediate segment and the proximal segment. A coil having a proximal end and a distal end is disposed about at least a portion of the distal segment of the elongate core member and disposed adjacent to the distal shoulder. A distal tip comprising a polymer material is disposed adjacent to the distal end of the coil and a proximal polymer member, the proximal polymer member disposed adjacent to the proximal shoulder. At least the intermediate segment and the coil have a diameter that is substantially the same.

### CROSS REFERENCE TO RELATED APPLICATIONS

This application claims the benefit of U.S. Provisional Patent Application Ser. No. 62/319,080 filed on Apr. 6, 2016, the disclosure of which is incorporated herein by reference.

### TECHNICAL FIELD

The present disclosure pertains to medical devices, and methods for manufacturing medical devices. More particularly, the present disclosure pertains to elongated intracorporeal medical devices including a tubular member connected with other structures, and methods for manufacturing and using such devices.

### BACKGROUND

A wide variety of intracorporeal medical devices have been developed for medical use, for example, intravascular use. Some of these devices include guidewires, catheters, and the like. These devices are manufactured by any one of a variety of different manufacturing methods and may be used according to any one of a variety of methods. Of the known medical devices and methods, each has certain advantages and disadvantages. There is an ongoing need to provide alternative medical devices as well as alternative methods for manufacturing and using medical devices.

### BRIEF SUMMARY

This disclosure provides design, material, manufacturing method, and use alternatives for medical devices. In one aspect, the disclosure invention relates to a guidewire including an elongate core member having an intermediate segment, a distal segment and a proximal segment, a distal shoulder between the intermediate segment and the distal segment and a proximal shoulder between the intermediate segment and the proximal segment. A coil having a proximal end and a distal end is disposed about at least a portion of the distal segment of the elongate core member and disposed adjacent to the distal shoulder. A distal tip comprising a polymer material is disposed adjacent to the distal end of the coil and a proximal polymer member, the proximal polymer member disposed adjacent to the proximal shoulder. At least the intermediate segment and the coil have a diameter that is substantially the same.

Alternatively or additionally to any of the embodiments above, the elongate core member comprises stainless steel.

Alternatively or additionally to any of the embodiments above, the elongate core member comprises a nickel-titanium alloy.

Alternatively or additionally to any of the embodiments above, the coil comprises a nickel-titanium alloy.

Alternatively or additionally to any of the embodiments above, the coil comprises stainless steel.

Alternatively or additionally to any of the embodiments above, the coil is a flat wire coil.

Alternatively or additionally to any of the embodiments above, the distal tip comprises a hydrophilic polymer material.

Alternatively or additionally to any of the embodiments above, the distal tip comprises a radiopaque marker material.

Alternatively or additionally to any of the embodiments above, the distal tip is a tungsten-filled radiopaque tip.

Alternatively or additionally to any of the embodiments above, the polymer member comprises a fluoropolymer.

Alternatively or additionally to any of the embodiments above, the guidewire further comprises a polymeric coating on the core member and the coil.

Alternatively or additionally to any of the embodiments above, the coating comprises fluoropolymer.

In another aspect, the disclosure relates to a guidewire comprising an elongate core member, the elongate core member comprising an intermediate segment, a distal segment including a distal taper, a proximal segment comprising a proximal taper, a distal step between the intermediate segment and the distal segment and a proximal step between the intermediate segment and the proximal segment. A proximal polymeric member is disposed along the proximal taper of the proximal segment and abutting the proximal shoulder. A coil having a proximal end and a distal end is disposed about a portion of the distal segment, the proximal end of the coil abutting the distal shoulder. A distal tip comprising a polymer material is disposed about a distal end of the distal taper and abutting the distal end of the coil. The intermediate segment and the coil have a diameter that is substantially the same.

Alternatively or additionally to any of the embodiments above, at least a portion of the proximal polymer member has a diameter that is substantially the same as the intermediate segment and the coil.

Alternatively or additionally to any of the embodiments above, at least a portion of the distal tip has a diameter that is substantially the same as the intermediate segment and the coil.

Alternatively or additionally to any of the embodiments above, the elongate core member and the coil comprise a material selected from the group consisting of shape memory metal alloys and metal alloys.

Alternatively or additionally to any of the embodiments above, the proximal polymer member comprises a heat shrink material.

Alternatively or additionally to any of the embodiments above, the core member between the proximal and distal shoulder and the coil are coated with lubricious polymer material.

In another aspect, the disclosure relates to a guidewire, the guidewire comprising an elongate core member, the elongate core member comprising an intermediate segment, a distal segment including a distal taper, a proximal segment comprising a proximal taper, a distal step between the intermediate segment and the distal segment, a proximal step between the intermediate segment and the proximal segment, a proximal polymeric member disposed along the proximal taper of the proximal segment and abutting the proximal shoulder, a polymeric reinforcing member having a proximal end and a distal end disposed about a portion of the distal segment, the proximal end of the polymeric reinforcing member abutting the distal shoulder, a distal tip comprising a polymer material disposed about a distal end of the distal taper and abutting the distal end of the polymeric reinforcing member and wherein the intermediate segment and the polymeric reinforcing member have a diameter that is substantially the same.

Alternatively or additionally to any of the embodiments above, at least a portion of the proximal polymer member has a diameter that is substantially the same as the intermediate segment and the polymeric reinforcing member.

Alternatively or additionally to any of the embodiments above, at least a portion of the distal tip has a diameter that is substantially the same as the intermediate segment and the polymeric reinforcing member.

Alternatively or additionally to any of the embodiments above, the elongate core member and comprise a material selected from the group consisting of shape memory metal alloys and metal alloys.

Alternatively or additionally to any of the embodiments above, the polymeric reinforcing member comprises a heat shrink material.

Alternatively or additionally to any of the embodiments above, the polymeric reinforcing member comprises a flared distal end, the flared distal end flows into a polymeric distal tip.

In another aspect, the disclosure relates to a method of making a guidewire comprising the steps of shaping an elongate core member to form a tapered proximal segment, an intermediate segment defined by a distal shoulder and a proximal shoulder, and a tapered distal segment, wherein the intermediate segment has a diameter that is larger than that of the tapered proximal segment and the tapered distal segment, covering the proximal tapered to the proximal shoulder of the intermediate segment by heat shrinking a polymer material thereon, disposing a coil having a proximal end and a distal end over at least a portion of the tapered distal segment wherein the proximal end of the coil abuts the distal shoulder of the intermediate member wherein the diameter of the intermediate member and the at least a portion of the tapered distal segment comprising the coil are substantially the same and disposing a polymeric distal tip over the tapered portion wherein the distal tip abuts the distal end of the coil.

Alternatively or additionally to any of the embodiments above, the method further comprises forming the tapered proximal segment and the tapered distal segment by grinding the elongate core member.

The above summary of some embodiments is not intended to describe each disclosed embodiment or every implementation of the present disclosure. The Figures, and Detailed Description, which follow, more particularly exemplify these embodiments.

### BRIEF DESCRIPTION OF THE DRAWINGS

The disclosure may be more completely understood in consideration of the following detailed description in connection with the accompanying drawings, in which:

FIG. 1 is a cross-sectional side view of a portion of an example medical device;

FIG. 2 is a cross-sectional side view of a portion of an example medical device;

FIG. 3 is a cross-sectional side view of a portion of an example medical device;

and

FIG. 4 is a schematic diagram of an example instrument for measuring the flexural load of an example guidewire.

While the disclosure is amenable to various modifications and alternative forms, specifics thereof have been shown by way of example in the drawings and will be described in detail. It should be understood, however, that the intention is not to limit the invention to the particular embodiments described. On the contrary, the intention is to cover all modifications, equivalents, and alternatives falling within the spirit and scope of the disclosure.

### DETAILED DESCRIPTION

For the following defined terms, these definitions shall be applied, unless a different definition is given in the claims or elsewhere in this specification.

All numeric values are herein assumed to be modified by the term “about,” whether or not explicitly indicated. The term “about” generally refers to a range of numbers that one of skill in the art would consider equivalent to the recited value (i.e., having the same function or result). In many instances, the terms “about” may include numbers that are rounded to the nearest significant figure.

The recitation of numerical ranges by endpoints includes all numbers within that range (e.g. 1 to 5 includes 1, 1.5, 2, 2.75, 3, 3.80, 4, and 5).

As used in this specification and the appended claims, the singular forms “a”, “an”, and “the” include plural referents unless the content clearly dictates otherwise. As used in this specification and the appended claims, the term “or” is generally employed in its sense including “and/or” unless the content clearly dictates otherwise.

It is noted that references in the specification to “an embodiment”, “some embodiments”, “other embodiments”, etc., indicate that the embodiment described may include one or more particular features, structures, and/or characteristics. However, such recitations do not necessarily mean that all embodiments include the particular features, structures, and/or characteristics. Additionally, when particular features, structures, and/or characteristics are described in connection with one embodiment, it should be understood that such features, structures, and/or characteristics may also be used connection with other embodiments whether or not explicitly described unless clearly stated to the contrary.

The following detailed description should be read with reference to the drawings in which similar elements in different drawings are numbered the same. The drawings, which are not necessarily to scale, depict illustrative embodiments and are not intended to limit the scope of the disclosure.

Guidewires are employed in a variety of medical procedures to facilitate the placement of medical devices during diagnostic or interventional procedures. One common used is for the placement of endourological instruments during diagnostic or interventional procedures. Desirably, the guidewires have a high flexural modulus for providing stiffness to the guidewire allowing the urologist the ability to better straighten tortuous anatomy and the ability to deliver heavier instrumentation. Sometimes, a high flexural modulus, however, can result in permanent kinking in the guidewire. When a guide wire kinks, the ability to track through anatomy may be reduced along with the ability to deliver endourological instruments.

This disclosure pertains to guidewires having improved kind resistance by increasing the diameter of at least a portion of the core wire and employing a flat wire coil wrapped this increases both the flexural modulus and provides kink resistance and flexibility to the guidewires for maneuvering through tortuous anatomy.

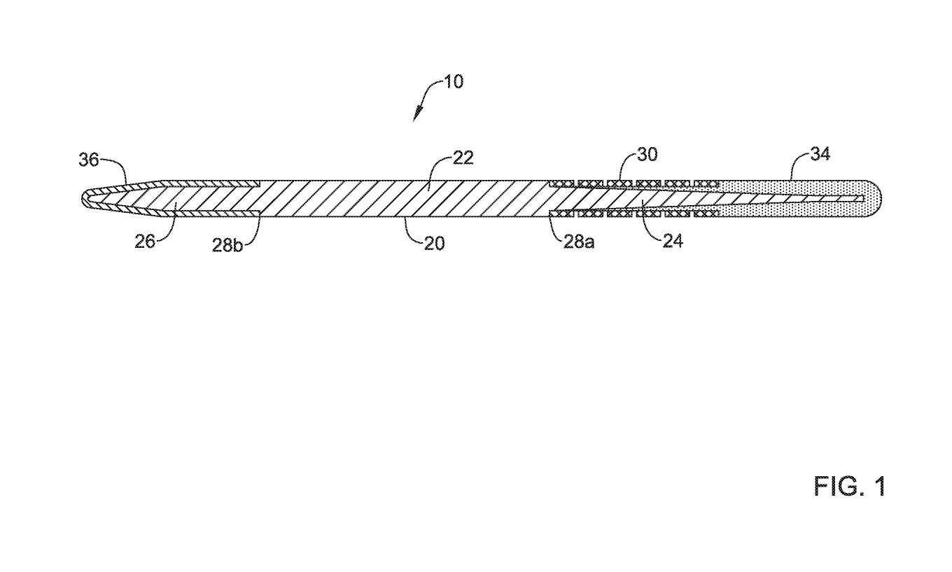

FIG. 1 is a cross-sectional side view of a portion of an example medical device, in this case, a guidewire 10. The guidewire 10 may include a core 20. The dimensions of the guide wire 10 and the core 20 will vary depending on the medical application.

Core 20 is shown having an intermediate portion 22, a distal portion 24 and a proximal portion 26. The intermediate portion 22 has a larger diameter than the distal portion 24 and the proximal portion 26. In between the intermediate portion 22 and the distal portion 24 is a distal step or shoulder 28a and in between the intermediate portion 22 and the proximal portion 26 is a proximal step or shoulder 28b. In some instances, the shoulders 28a/28b may be a step in diameter. As used herein the term “step” refers to a more vertical or perpendicular adjustment in diameter of the core 20 of the guidewire whereas the term “taper” refers to a more gradual incline or angle in the adjustment of the diameter of the core 20 of the guidewire. In other instances, the shoulders 28a/28b may include one or more tapers and/or steps.

Core 20 may be formed of spring steels, stainless steel, super-elastic materials such as the NiTi alloys e.g. NITINOL, linear-elastic materials, or other biocompatible materials including those disclosed herein. These materials can provide kink resistance. A guidewire 10 having a core 20 with a longer intermediate portion 22 and a shorter coil 30 can provide increased stiffness to the guidewire for improved deliverability.

In some embodiments, the core 20 is formed from a super-elastic material, for example, NiTi alloy.

The distal portion 24 of core 20 may be tapered, as shown, to provide flexibility to guide wire 10. Tapering can be accomplished using any suitable technique and in some embodiments, is accomplished by grinding.

Surrounding a portion of the distal portion 24 of core 20 is coil wire 30. Coil wire 30 may be a flat ribbon coil, but may also have a round or spherical shape, for example. Coil wire 30 may also include a coating of a more lubricious material such as a fluoropolymer coating, for example polytetrafluoroethylene. Coil wire 30 may also have an open or a closed pitch along the length or some combination thereof. Coil wire 30 may be made of a variety of metallic materials including super-elastic or linear-elastic materials such as NiTi alloy or NITINOL, stainless steel alloys such as 304V or 316L, or other suitable materials such as those disclosed herein.

Coil wire 30 is shown disposed about the distal portion 24 and adjacent to or abutting the distal step or shoulder 28a and with the distal portion 24 over which coil wire is disposed, has a diameter that is substantially the same as that of the intermediate portion 22.

For example, the guidewire 10 may have a diameter at the intermediate portion 22 and the distal portion 24 having the coil wire 30 disposed thereon may have a diameter of about 0.030″ to about 0.040″, for example 0.035″ or 0.038″.

Coil wire 30 is shown wrapped in a helical fashion about distal portion 24 of the core 20. The pitch chosen to wind the coil wire 30 may be determined by the particular application and flexibility requirements for the guide wire 10.

Coil wire 30 may be formed of round wire or of flat ribbon wire. The flat ribbon wire can be formed by, for example, rolling a round cross sectional wire. The transverse ends of the cross section of the ribbon wire can be rounded or with subsequent processing squared. It can be appreciated that numerous cross sectional shapes can be used in accordance with the present invention. Or, coil 30 may be formed of cross-wound multifilar or multifilar single coil wire.

In some embodiments, the coil wire 30 is a flat wire coil and in some embodiments, the coil 30 is a stainless steel flat wire coil.

The pitch can vary from tightly wrapped so that each turn touches the preceding turn or the pitch may be such that coil wire 30 is wrapped about the distal portion 24 of the core 20 in an open fashion so that there is space between each succeeding turn of the coil wire. Succeeding wraps 35 of coil wire 30 may or may not overlap or touch the preceding wrap. Guidewires having wire coils of this type are disclosed in commonly assigned U.S. Pat. No. 6,494,894 the entire content of which is incorporated by reference herein.

The remaining portion of the distal taper 24 may surrounded by a distal polymer tip 34 which may be adjacent to or abut the distal end of the coil 30. In some instances, the polymer tip 34 may extend underneath at least a portion of the coil 30 and, in some cases, may abut or extend to a position adjacent to shoulder 28a. The distal polymer tip 34 may be coated by or formed of a hydrophilic material. A hydrophilic distal tip can facilitate passage of obstructions in the body lumens of a patient such as an impacted stone, for example.

The distal tip 34 may also include a radiopaque marker material. A suitable radiopaque material can be employed as is well known in the art. In some embodiments, the distal polymer tip may be a tungsten filled radiopaque tip for enhanced fluoroscopic visualization.

In some embodiments, at least a portion of the distal portion 24 that is adjacent to or abuts the distal end of the coil 30, has a diameter that is substantially the same as that of the intermediate portion 22 and the distal portion 24 having the coil 30 disposed thereon.

The proximal portion 26 of the core wire 20 may also be surrounded by a proximal polymer member 36. This polymer member 36 may have a tapered proximal portion. Tapering of the proximal portion 26 can facilitate introduction of instruments over the guidewire 10. The proximal polymer member 36 may be adjacent to or abut the proximal shoulder or step 28b at the intermediate portion 22.

Tapering of the proximal portion 26 of the core wire can also be accomplished using any suitable method such as, for example, grinding.

The proximal polymer member 36 can be formed of any suitable polymer material and, in some embodiments, the proximal polymer member 36 is formed from a heat shrink material such as a fluoropolymer, e.g. polytetrafluoroethylene for providing lubricity to the proximal polymer member 36. The proximal polymer member 36 may abut the proximal shoulder 28b.

In some embodiments, at least a portion of the proximal portion 26 having the proximal polymer member 36 that is adjacent to the intermediate portion 22 may have a diameter that is substantially the same as that of the intermediate portion 22 and the distal portion 24 having the coil 30 disposed thereon.

The intermediate portion 22 of the core 20 and the coil 30 may be coated with a lubricious material such as with a hydrophobic material, for example a fluoropolymer, e.g. polytetrafluoroethylene or silicon, or a hydrophilic materials such as HYDROPASS. Lubricious coatings may facilitate advancement and delivering of the guidewire 10. Lubricious coatings can also facilitate the introduction of other medical devices during delivery over the guidewire 10.

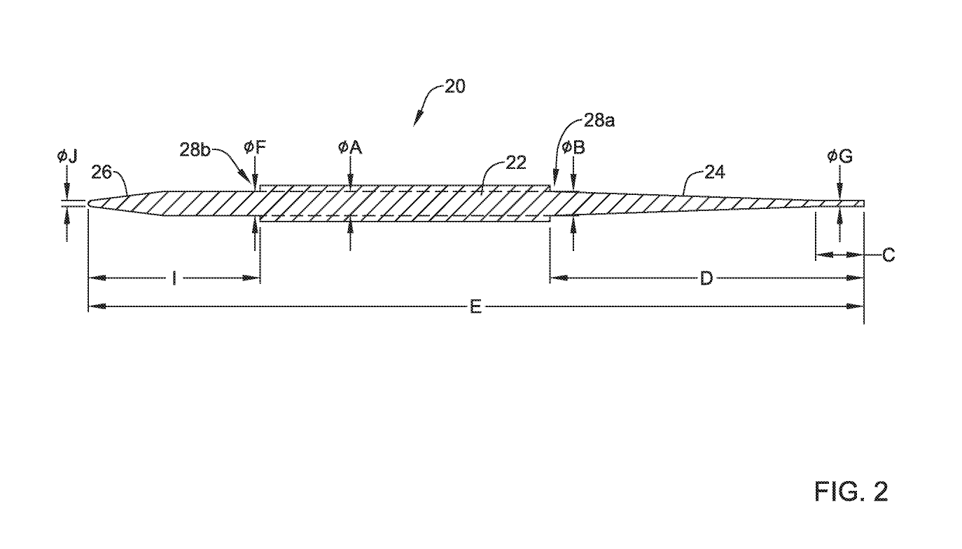

FIG. 2 is a side cross-sectional view of an example core wire 20 having a length E. Core wire 20 is shown having an intermediate portion 22 having a diameter A that has a larger diameter than that of either the distal portion 24 having a length D or the proximal portion 26 having a length I. In between the distal portion 24 and the intermediate portion 22 is a distal shoulder or step 28a having a diameter F that is less than that of the intermediate portion and in between the proximal portion 26 and the intermediate portion 22 is a proximal shoulder or step 28b having a diameter B that is also less than that of the intermediate portion 22. Each of the proximal end of the proximal portion 26 and the distal end of the distal portion 24 are shown having a taper which ends at a diameter J and G respectively. Distal portion 24 may terminate in a constant diameter section having a length C.

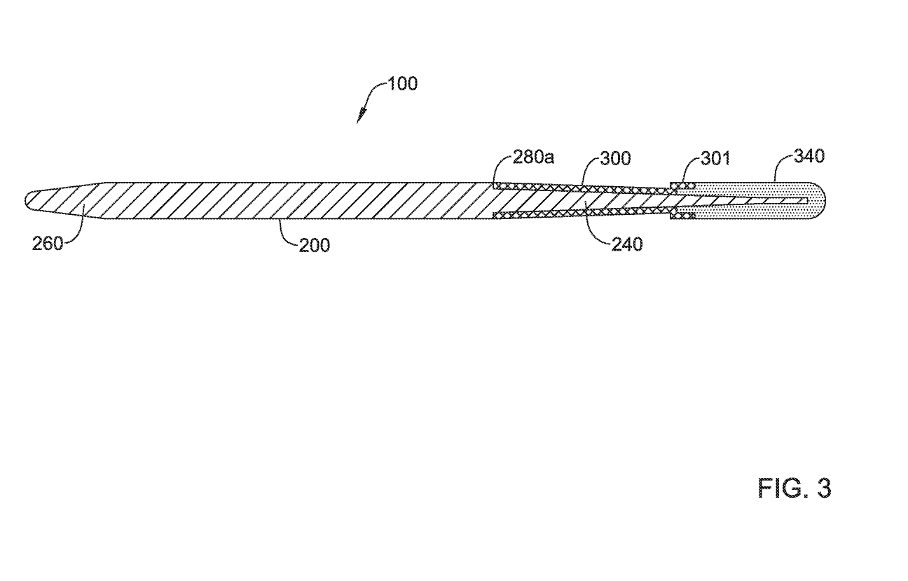

FIG. 3 is a side cross-sectional view of an example guidewire 100 having core wire 200. A reinforcing member 300 abuts shoulder or step 280a. In this example, reinforcing member 300 is in the form of a polymeric member, for example, a heat shrink polymer having a flared distal end 301. The flared end 301 may flow into or otherwise about a portion of a distal tip 340.

The reinforcing member may be formed of fluoropolymers such as fluorinated ethylene propylene (FEP), polytetrafluoroethylene (PTFE) and expanded polytetrafluoroethylene (EPTFE), terpolymers of ethylene tetrafluoroethylene and hexafluoropropylene (EFEP), and combinations thereof.

In some embodiments, a removable heat shrink processing aid can be employed to aid in reflowing of the polymeric reinforcing member the polymeric reinforcing extrusion, for example, with an EFEP or FEP extrusion on the distal wire section. For example, a temporary heat shrink tube can be disposed about the polymeric reinforcing member prior to the reflow process (e.g., in order to aid the reflow process) and removed following the reflow process.

The above lists and example materials are intended only for illustrative purposes and not as a limitation on the scope of the present disclosure.

### EXAMPLE

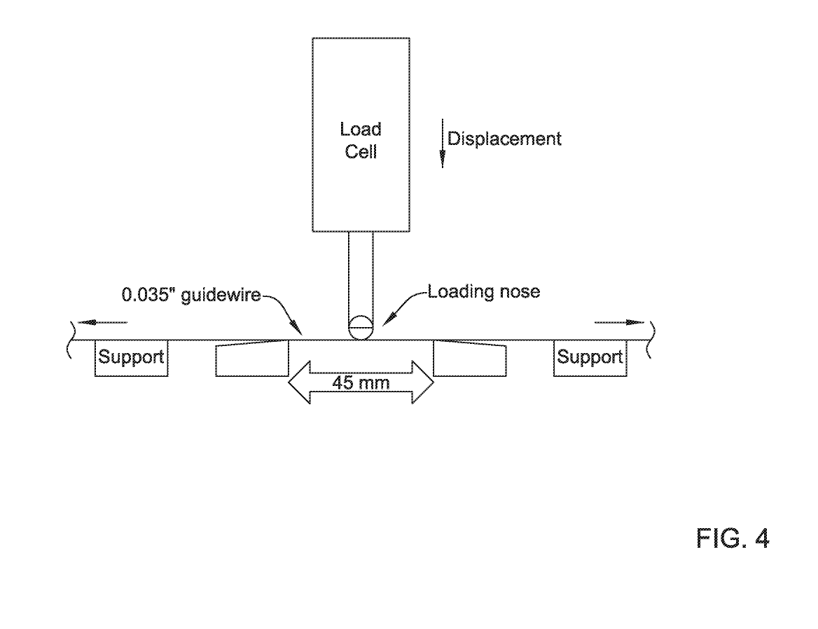

The stiffness of a guidewire as formed substantially as disclosed herein and having a nitinol core wire and a stainless steel coil having a diameter of 0.035″ as disclosed herein, may be measured utilizing a 3 point bend test method by compressing down a guidewire that is supported on either end at a fixed distance. The force exerted back is measured by a load cell and is referred to as flexural load as shown in FIG. 4.

The test parameters employed include the following:
Semicircular Angle Loading nose was used
Supports were set at 45 mm apart.
Nose was displaced down at a constant speed of 20 mm/min
Load cell was either a 50 or 100N cell
Maximum deflection was set to 5 mm
Because of the long length of the guidewire, 9-10 separate points were measured along the length of the guidewire. The first 40-60 cm from the distal end was not tested. This was due to avoid testing the floppy tip.

The force required to deflect a wire 5 mm for a 0.035″ guidewire was about 5.80 Newtons. For a 0.038″ guidewire, the force required to deflect the wire 5 mm was about 8.0 Newtons.

The guidewires disclosed herein can be employed in any noninvasive medical procedure for delivery of medical devices therein. As an example, the guidewires disclosed herein can be employed in urological procedures to facilitate the placement of endourological instruments during diagnostic or interventional procedures. The wires disclosed herein have superior stiffness and flexural modulus for improved ability to straighten tortuous anatomy and improved ability to deliver heavier instruments.

The materials that can be used for guidewire 10 and/or the various components thereof (and/or other guidewires/components disclosed/contemplated herein) may include those commonly associated with medical devices. For example, guidewire 10 and/or other components of guidewire 10 may be made from a metal, metal alloy, polymer (some examples of which are disclosed below), a metal-polymer composite, ceramics, combinations thereof, and the like, or other suitable material. Some examples of suitable polymers may include polytetrafluoroethylene (PTFE), ethylene tetrafluoroethylene (ETFE), fluorinated ethylene propylene (FEP), polyoxymethylene (POM, for example, DELRIN® available from DuPont), polyether block ester, polyurethane (for example, Polyurethane 85A), polypropylene (PP), polyvinylchloride (PVC), polyether-ester (for example, ARNITEL® available from DSM Engineering Plastics), ether or ester based copolymers (for example, butylene/poly(alkylene ether) phthalate and/or other polyester elastomers such as HYTREL® available from DuPont), polyamide (for example, DURETHAN® available from Bayer or CRISTAMID® available from Elf Atochem), elastomeric polyamides, block polyamide/ethers, polyether block amide (PEBA, for example available under the trade name PEBAX®), ethylene vinyl acetate copolymers (EVA), silicones, polyethylene (PE), Marlex high-density polyethylene, Marlex low-density polyethylene, linear low density polyethylene (for example REXELL®), polyester, polybutylene terephthalate (PBT), polyethylene terephthalate (PET), polytrimethylene terephthalate, polyethylene naphthalate (PEN), polyetheretherketone (PEEK), polyimide (PI), polyetherimide (PEI), polyphenylene sulfide (PPS), polyphenylene oxide (PPO), poly paraphenylene terephthalamide (for example, KEVLAR®), polysulfone, nylon, nylon-12 (such as GRILAMID® available from EMS American Grilon), perfluoro(propyl vinyl ether) (PFA), ethylene vinyl alcohol, polyolefin, polystyrene, epoxy, polyvinylidene chloride (PVdC), poly(styrene-b-isobutylene-b-styrene) (for example, SIBS and/or SIBS 50A), polycarbonates, ionomers, biocompatible polymers, other suitable materials, or mixtures, combinations, copolymers thereof, polymer/metal composites, and the like. In some embodiments the sheath can be blended with a liquid crystal polymer (LCP). For example, the mixture can contain up to about 6 percent LCP.

Some examples of suitable metals and metal alloys include stainless steel, such as 304V, 304L, and 316LV stainless steel; mild steel; nickel-titanium alloy such as linear-elastic and/or super-elastic nitinol; other nickel alloys such as nickel-chromium-molybdenum alloys (e.g., UNS: N06625 such as INCONEL® 625, UNS: N06022 such as HASTELLOY® C-22®, UNS: N10276 such as HASTELLOY® C276®, other HASTELLOY® alloys, and the like), nickel-copper alloys (e.g., UNS: N04400 such as MONEL® 400, NICKELVAC® 400, NICORROS® 400, and the like), nickel-cobalt-chromium-molybdenum alloys (e.g., UNS: R30035 such as MP35-N® and the like), nickel-molybdenum alloys (e.g., UNS: N10665 such as HASTELLOY® ALLOY B2®), other nickel-chromium alloys, other nickel-molybdenum alloys, other nickel-cobalt alloys, other nickel-iron alloys, other nickel-copper alloys, other nickel-tungsten or tungsten alloys, and the like; cobalt-chromium alloys; cobalt-chromium-molybdenum alloys (e.g., UNS: R30003 such as ELGILOY®, PHYNOX®, and the like); platinum enriched stainless steel; titanium; combinations thereof; and the like; or any other suitable material.

In at least some embodiments, portions or all of guidewire 10 may also be doped with, made of, or otherwise include a radiopaque material. Radiopaque materials are understood to be materials capable of producing a relatively bright image on a fluoroscopy screen or another imaging technique during a medical procedure. This relatively bright image aids the user of guidewire 10 in determining its location. Some examples of radiopaque materials can include, but are not limited to, gold, platinum, palladium, tantalum, tungsten alloy, polymer material loaded with a radiopaque filler, and the like. Additionally, other radiopaque marker bands and/or coils may also be incorporated into the design of guidewire 10 to achieve the same result.

In some embodiments, a degree of Magnetic Resonance Imaging (MRI) compatibility is imparted into guidewire 10. For example, guidewire 10, or portions thereof, may be made of a material that does not substantially distort the image and create substantial artifacts (e.g., gaps in the image). Certain ferromagnetic materials, for example, may not be suitable because they may create artifacts in an MRI image. Guidewire 10, or portions thereof, may also be made from a material that the MRI machine can image. Some materials that exhibit these characteristics include, for example, tungsten, cobalt-chromium-molybdenum alloys (e.g., UNS: R30003 such as ELGILOY®, PHYNOX®, and the like), nickel-cobalt-chromium-molybdenum alloys (e.g., UNS: R30035 such as MP35-N® and the like), nitinol, and the like, and others.

It should be understood that this disclosure is, in many respects, only illustrative. Changes may be made in details, particularly in matters of shape, size, and arrangement of steps without exceeding the scope of the disclosure. This may include, to the extent that it is appropriate, the use of any of the features of one example embodiment being used in other embodiments. The invention's scope is, of course, defined in the language in which the appended claims are expressed.

1. A guidewire comprising:
an elongate core member having an intermediate segment, a distal segment and a proximal segment, a distal shoulder between the intermediate segment and the distal segment and a proximal shoulder between the intermediate segment and the proximal segment;
a coil having a proximal end and a distal end disposed about at least a portion of the distal segment of the elongate core member and disposed adjacent to the distal shoulder;
a distal tip comprising a polymer material, the distal tip disposed adjacent to the distal end of the coil; and
a proximal polymer member, the proximal polymer member disposed adjacent to the proximal shoulder; and
wherein at least the intermediate segment and the coil have a diameter that is substantially the same.

2. The guidewire of claim 1 wherein the elongate core member comprises stainless steel.

3. The guidewire of claim 2 wherein the elongate core member comprises a nickel-titanium alloy.

4. The guidewire of claim 1 wherein the coil comprises a nickel-titanium alloy.

5. The guidewire of claim 4 wherein the coil comprises stainless steel.

6. The guidewire of claim 1 wherein the coil is a flat wire coil.

7. The guidewire of claim 1 wherein the distal tip further comprises a coating of a hydrophilic polymer material.

8. The guidewire of claim 1 wherein the distal tip comprises a radiopaque marker material.

9. The guidewire of claim 1 wherein the distal tip is a tungsten-filled radiopaque tip.

10. The guidewire of claim 1 wherein the polymer member comprises a fluoropolymer.

11. The guidewire of claim 1 further comprising a polymeric coating on the core member and the coil.

12. The guidewire of claim 11 wherein the coating comprises fluoropolymer.

13. A guidewire, the guidewire comprising an elongate core member, the elongate core member comprising:
an intermediate segment;
a distal segment including a distal taper;
a proximal segment comprising a proximal taper;
a distal step between the intermediate segment and the distal segment;
a proximal step between the intermediate segment and the proximal segment;
a proximal polymeric member disposed along the proximal taper of the proximal segment and abutting the proximal shoulder;
a polymeric reinforcing member having a proximal end and a distal end disposed about a portion of the distal segment, the proximal end of the polymeric reinforcing member abutting the distal shoulder;
a distal tip comprising a polymer material disposed about a distal end of the distal taper and abutting the distal end of the polymeric reinforcing member; and
wherein the intermediate segment and the polymeric reinforcing member have a diameter that is substantially the same.

14. The guidewire of claim 13 wherein at least a portion of the proximal polymer member has a diameter that is substantially the same as the intermediate segment and the polymeric reinforcing member.

15. The guidewire of claim 13 wherein at least a portion of the distal tip has a diameter that is substantially the same as the intermediate segment and the polymeric reinforcing member.

16. The guidewire of claim 13 wherein the elongate core member comprises a material selected from the group consisting of shape memory metal alloys and metal alloys.

17. The guidewire of claim 13 wherein the polymeric reinforcing member comprises a heat shrink material.

18. The guidewire of claim 13 wherein the polymeric reinforcing member comprises a flared distal end, the flared distal end flows into the distal tip.

19. A method of making a guidewire comprising the steps of:
shaping an elongate core member to form a tapered proximal segment, an intermediate segment defined by a distal shoulder and a proximal shoulder, and a tapered distal segment, wherein the intermediate segment has a diameter that is larger than that of the tapered proximal segment and the tapered distal segment;
covering the proximal tapered to the proximal shoulder of the intermediate segment by heat shrinking a polymer material thereon;
disposing a coil having a proximal end and a distal end over at least a portion of the tapered distal segment wherein the proximal end of the coil abuts the distal shoulder of the intermediate member wherein the diameter of the intermediate member and the at least a portion of the tapered distal segment comprising the coil are substantially the same; and
disposing a polymeric distal tip over the tapered portion wherein the distal tip abuts the distal end of the coil.

20. The method of claim 19 further comprising forming the tapered proximal segment and the tapered distal segment by grinding the elongate core member.
